(19) United States
(12) Patent Application Publication
(10) Pub. No.: US 2023/0218894 A1
(43) Pub. Date: Jul. 13, 2023

(54) IMPLANTABLE CANCER THERAPY ELECTRODES WITH REDUCED MRI ARTIFACTS

(71) Applicant: Cardiac Pacemakers, Inc., St. Paul, MN (US)

(72) Inventors: Devon N. Arnholt, Shoreview, MN (US); Michael J. Kane, St. Paul, MN (US)

(73) Assignee: MAYO FOUNDATION FOR MEDICAL EDUCATION AND RESEARCH, Rochester, MN (US)

(21) Appl. No.: 18/094,618

(22) Filed: Jan. 9, 2023

Related U.S. Application Data
(60) Provisional application No. 63/298,528, filed on Jan. 11, 2022.

(51) Int. Cl. A61N 1/08; A61N 1/36

(52) U.S. Cl. CPC A61N 1/086; A61N 1/36002

(57) ABSTRACT

Embodiments herein relate to implantable cancer therapy electrodes with reduced magnetic resonance imaging artifacts. In an embodiment, a lead for a cancer treatment system can include a lead body with a proximal end and a distal end and defining a lumen, and one or more electric field generating electrodes, wherein the one or more electric field generating electrodes can be disposed along a length of the lead body. The one or more electric field generating electrodes include a ribbon wire with a thickness of the ribbon wire in a radial direction with respect to the lead body of less than 0.005 inches, or a walled tube with a thickness of the walled tube less than 0.005 inches, or a sputter coating with a thickness of the sputter coating in a radial direction with respect to a lead body of less than 0.005 inches. Other embodiments are also included herein.

This application claims the benefit of U.S. Provisional Application No. 63/298,528, filed Jan. 11, 2022, the content of which is herein incorporated by reference in its entirety.

### FIELD

Embodiments herein relate to implantable cancer therapy electrodes with reduced magnetic resonance imaging artifacts.

### BACKGROUND

Monitoring cancerous tumor growth in a patient during the course of a cancer treatment can provide insights to medical personnel about the effectiveness the treatment. Magnetic resonance imaging (MRI) is one technique that can be used to monitor the progression or regression of a cancerous tumor in response to a given therapy regimen.

When subjected to an external magnetic field applied by an MRI device, the metal components of an implantable device can be unsafe for a patient due to localized heating caused by the ferromagnetic nature of many metals present in such devices. The presence of metals in implanted medical devices can also contribute to artifacts in MRI signals collected to monitor a course of treatment, leading to visual distortion of the images obtained at or near the site of the implantable medical device components. MRI artifacts in data signals can confound the data analysis process when determining progression or regression of a cancerous tumor.

### SUMMARY

Embodiments herein relate to implantable cancer therapy electrodes with reduced magnetic resonance imaging artifacts. In a first aspect, a lead for a cancer treatment system can be included having a lead body with a proximal end and a distal end, the lead body defining a lumen, and one or more electric field generating electrodes, wherein the one or more electric field generating electrodes can be disposed along a length of the lead body, wherein the one or more electric field generating electrodes include a ribbon wire, wherein a thickness of the ribbon wire in a radial direction with respect to the lead body can be less than 0.005 inches, or a walled tube, wherein a thickness of the walled tube can be less than 0.005 inches, or a sputter coating, wherein a thickness of the sputter coating in a radial direction with respect to a lead body can be less than 0.005 inches.

In a second aspect, in addition to one or more of the preceding or following aspects, or in the alternative to some aspects, a thickness of the ribbon wire in a radial direction with respect to the lead body can be less than 0.005 inches and greater than 0.00001 inches.

In a third aspect, in addition to one or more of the preceding or following aspects, or in the alternative to some aspects, a thickness of the walled tube can be less than 0.005 inches and greater than 0.00001 inches.

In a fourth aspect, in addition to one or more of the preceding or following aspects, or in the alternative to some aspects, the one or more electric field generating electrodes can be configured to reduce metal-induced magnetic resonance imaging artifacts.

In a fifth aspect, in addition to one or more of the preceding or following aspects, or in the alternative to some aspects, the one or more electric field generating electrodes include a ribbon wire, the ribbon wire having an aspect ratio between a thickness and a width of the ribbon of from 2:4 to 2:20.

In a sixth aspect, in addition to one or more of the preceding or following aspects, or in the alternative to some aspects, the one or more electric field generating electrodes include a ribbon wire, the ribbon wire having an aspect ratio between a thickness and a width of the ribbon of from 2:8 to 2:10.

In a seventh aspect, in addition to one or more of the preceding or following aspects, or in the alternative to some aspects, the system can include two electric field generating electrodes disposed along a length of the lead body.

In an eighth aspect, in addition to one or more of the preceding or following aspects, or in the alternative to some aspects, the two electric field generating electrodes can be each independently from 1 cm to 4 cm in length.

In a ninth aspect, in addition to one or more of the preceding or following aspects, or in the alternative to some aspects, the two electric field generating electrodes can be each independently 2 cm in length.

In a tenth aspect, in addition to one or more of the preceding or following aspects, or in the alternative to some aspects, the two electric field generating electrodes can be separated by a non-conducting gap portion of from 0.1 cm to 2 cm.

In an eleventh aspect, in addition to one or more of the preceding or following aspects, or in the alternative to some aspects, the one or more electric field generating electrodes include one or more of copper, aluminum, silver, platinum, titanium, nickel, or a metal alloy.

In a twelfth aspect, in addition to one or more of the preceding or following aspects, or in the alternative to some aspects, the ribbon wire or the walled tube can include a helical conductor pattern.

In a thirteenth aspect, in addition to one or more of the preceding or following aspects, or in the alternative to some aspects, the helical conductor pattern can have an outside diameter of 1 to 3 millimeters.

In a fourteenth aspect, in addition to one or more of the preceding or following aspects, or in the alternative to some aspects, the one or more electric field generating electrodes can include from one to six conductive paths arranged in parallel.

In a fifteenth aspect, a method of treating a patient previously diagnosed with cancer can be included, the method can include implanting a first lead within a patient.

The first lead can include a first lead body can include a proximal end and a distal end, and one or more electric field generating electrodes disposed along a length of the lead body, wherein the one or more electric field generating electrodes include a ribbon wire, wherein a thickness of the ribbon wire in a radial direction with respect to the lead body can be less than 0.005 inches, or a walled tube, wherein a thickness of the walled tube can be less than 0.005 inches, or a sputter coating, and wherein a thickness of the sputter coating in a radial direction with respect to the lead body can be less than 0.005 inches. positioning the lead at or near a site of a cancerous tumor. The method can also include generating one or more electric fields with the one or more electric field generating electrodes.

In a sixteenth aspect, in addition to one or more of the preceding or following aspects, or in the alternative to some aspects, the method can further include implanting a second lead within a patient.

In a seventeenth aspect, in addition to one or more of the preceding or following aspects, or in the alternative to some aspects, the second lead includes a second lead body can include a proximal end and a distal end, and wherein the one or more electric field generating electrodes include a ribbon wire, wherein a thickness of the ribbon wire in a radial direction with respect to the lead body can be less than 0.005 inches, or a walled tube, and wherein a thickness of the walled tube can be less than 0.005 inches.

In an eighteenth aspect, in addition to one or more of the preceding or following aspects, or in the alternative to some aspects, generating one or more electric fields with the one or more electrodes includes generating an electrical field at a treatment site having a field strength of between 1 V/cm to 10 V/cm.

In a nineteenth aspect, in addition to one or more of the preceding or following aspects, or in the alternative to some aspects, the one or more electric fields can be delivered at one or more frequencies selected from a range of between 100 kHz to 300 kHz.

In a twentieth aspect, in addition to one or more of the preceding or following aspects, or in the alternative to some aspects, the one or more electric fields can be generated using currents ranging from 20 mAmp to 500 mAmp.

In a twenty-first aspect, in addition to one or more of the preceding or following aspects, or in the alternative to some aspects, the one or more electrodes forming electrode pairs can define at least two different electrical field vectors.

In a twenty-second aspect, in addition to one or more of the preceding or following aspects, or in the alternative to some aspects, the two different electrical field vectors can be angled by at least 10 degrees with respect to one another.

In a twenty-third aspect, in addition to one or more of the preceding or following aspects, or in the alternative to some aspects, the method can further include obtaining magnetic resonance images of a cancerous tumor to determine a progression or regression of the cancerous tumor.

In a twenty-fourth aspect, a medical device system for treating a cancerous tissue can be included having an electric field generating circuit configured to generate one or more electric fields at or near a site of the cancerous tissue, control circuitry in communication with the electric field generating circuit, the control circuitry configured to control delivery of the one or more electric fields from the electric field generating circuit to the site of the cancerous tissue, and an implantable first lead, the implantable first lead can include a lead body having a proximal end and a distal end, the lead body defining a lumen, and one or more electric field generating electrodes disposed along a length of the lead body. The one or more electric field generating electrodes can include a ribbon wire. A thickness of the ribbon wire in a radial direction with respect to the lead body can be less than 0.005 inches. The one or more electric field generating electrodes can also or alternatively include a walled tube, wherein a thickness of the walled tube can be less than 0.005 inches. The one or more electric field generating electrodes can also include a sputter coating, wherein a thickness of the sputter coating in a radial direction with respect to the lead body can be less than 0.005 inches.

In a twenty-fifth aspect, in addition to one or more of the preceding or following aspects, or in the alternative to some aspects, the medical device system can further include an implantable second lead, the implantable second lead can include a lead body having a proximal end and a distal end, the lead body defining a lumen, and one or more electric field generating electrodes disposed along a length of the lead body. The one or more electric field generating electrodes include a ribbon wire, wherein a thickness of the ribbon wire in a radial direction with respect to the lead body can be less than 0.005 inches, or a walled tube, wherein a thickness of the walled tube can be less than 0.005 inches, or a sputter coating, wherein a thickness of the sputter coating in a radial direction with respect to the lead body can be less than 0.005 inches.

In a twenty-sixth aspect, in addition to one or more of the preceding or following aspects, or in the alternative to some aspects, the control circuitry causes the electric field generating circuit to generate one or more electric fields at frequencies selected from a range of between 10 kHz to 1 MHz.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a medical system in accordance with various embodiments herein.

FIG. 2 is a schematic view of a medical system in accordance with various embodiments herein.

FIG. 3 is a schematic view of a medical system in accordance with various embodiments herein.

FIG. 4 is a schematic view of a medical system in accordance with various embodiments herein.

FIG. 5 is a schematic view of a cancer therapy stimulation lead in accordance with various embodiments herein.

FIG. 6 is a cross-sectional view of the lead in FIG. 5 along line 6-6′ in accordance with various embodiments herein.

FIG. 7 is a perspective view of a cancer therapy stimulation lead in accordance with various embodiments herein.

FIG. 8 is a perspective view of a deconstructed cancer therapy stimulation lead in accordance with various embodiments herein.

FIG. 9 is a cross-sectional schematic view of a portion of a lead including an electrode in accordance with various embodiments herein.

FIG. 10 is a cross-sectional schematic view of a portion of a lead including an electrode in accordance with various embodiments herein.

FIG. 11 is a cross-sectional schematic view of a portion of a lead including an electrode in accordance with various embodiments herein.

FIG. 12 is a cross-sectional schematic view of a portion of a lead including an electrode in accordance with various embodiments herein.

FIG. 13 is a schematic view of a portion of a lead including an electrode in accordance with various embodiments herein.

FIG. 14 is a schematic view of a portion of a lead including an electrode in accordance with various embodiments herein.

FIG. 15 is a schematic view of a portion of a lead including an electrode in accordance with various embodiments herein.

FIG. 16 is a schematic view of a portion of a lead including an electrode in accordance with various embodiments herein.

FIG. 17 is a cross-sectional schematic view of a portion of a lead including an electrode in accordance with various embodiments herein.

FIG. 18 is a cross-sectional schematic view of the electrode of FIG. 17 along line 18-18′ in accordance with various embodiments herein.

FIG. 19 is a cross-sectional schematic view of a portion of a lead including an electrode in accordance with various embodiments herein.

FIG. 20 is a cross-sectional schematic view of a portion of a lead including an electrode in accordance with various embodiments herein.

FIG. 21 is a schematic view of a medical device in accordance with various embodiments herein.

FIG. 22 is a plot of an exemplary therapy parameter in accordance with various embodiments herein.

FIG. 23 is a plot of an exemplary therapy parameter in accordance with various embodiments herein.

FIG. 24 is a schematic cross-sectional view of a medical device in accordance with various embodiments herein.

FIG. 25 is a schematic diagram of components of a medical device in accordance with various embodiments herein.

FIG. 26 is a flow chart depicting a method in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein include implantable leads and implantable electric field generating electrodes that can be used as part of a medical device and medical device system. The treatment of various cancerous tumors with implantable leads and implantable electric field generating electrodes can be monitored with various imaging techniques to determine the progression or regression of a cancerous tumor. The use of magnetic resonance imaging (MRI) can serve as a diagnostic imaging tool during a course of treatment for a cancerous tumor. However, as discussed above, MRI artifacts caused by the presence of metal in the applied magnetic fields emitted by an MRI device can confound the ability of a medical practitioner to accurately analyze the MRI data and can result localized heating within a patient during an MRI procedure.

Without wishing to be bound by any particular theory, it is believed that reducing the thickness of metal associated with an electrode at or near the site of a cancerous tumor can greatly reduce the MRI artifacts in the MRI signal data. Various electrode structures are described herein that are configured to reduce the thickness of metal in the electrode, and thus reduce the extent of the MRI artifact at the cancerous tumor site to allow for more accurate visualization of the tumor structures near the implantable electrodes. The electric field generating electrodes herein can include those made from one or more thin-walled metallic structures including wire ribbons, walled tubes, or sputtered metallic coatings to reduce the thickness of metal at or near the site of a cancerous tumor in order to allow for enhance visualization of the cancerous tumor during treatment.

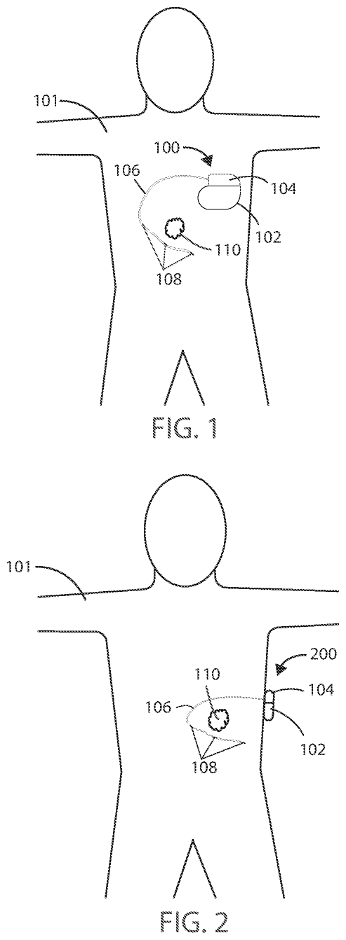

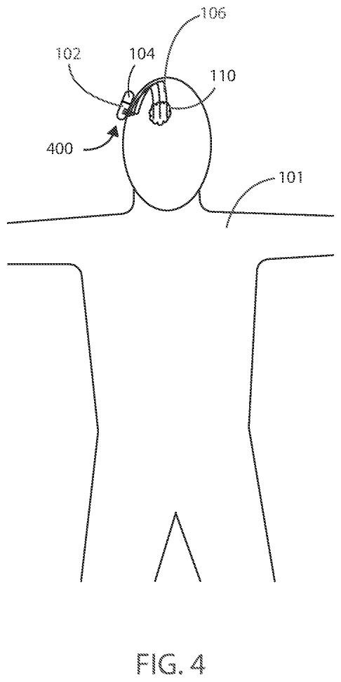

Various ribbon wire and walled tube electrodes can be used in implantable leads and medical device systems as described herein to treat a cancerous tumor and to reduce MRI artifacts when visualizing the cancerous tumor during diagnostic and monitoring procedures. Referring now to FIG. 1 and FIG. 2, schematic diagrams of a patient 101 with a cancerous tumor 110 are shown in accordance with the embodiments herein. In FIG. 1, the patient 101 has a medical device 100 implanted entirely within the body of the patient 101 at or near the site of cancerous tumor 110. Various implant sites can be used including areas such as in the limbs, the upper torso, the abdominal area, the head, and the like. In FIG. 4, the patient 101 has a medical device 400 at least partially implanted within body of the patient 101 at or near the site of a cancerous tumor. In some embodiments, the medical device can be entirely external to the subject. In some embodiments, the medical device can be partially external to the subject. In some embodiments, the medical device can be partially implanted and partially external to the body of a subject. In other embodiments, a partially implanted medical device can include a transcutaneous connection between components disposed internal to the body and external to the body. A partially or fully implanted medical device can wirelessly communicate with a partially or fully external portion of a medical device over a wireless connection.

In some embodiments, a portion of the medical device can be entirely implanted, and a portion of the medical device can be entirely external. For example, in some embodiments, one or more electrodes or leads can be entirely implanted within the body, whereas the portion of the medical device that generates an electric field, such as an electric field generator, can be entirely external to the body. It will be appreciated that in some embodiments described herein, the electric field generators described can include many of the same components and can be configured to perform many of the same functions, as a pulse generator. In embodiments where a portion of a medical device is entirely implanted, and a portion of the medical device is entirely external, the portion of the medical device that is entirely external can communicate wirelessly with the portion of the medical device that is entirely internal. However, in other embodiments a wired connection can be used.

The medical device 100 and medical device 200 can each include a housing 102 and a header 104 coupled to the housing 102. Various materials can be used. However, in some embodiments, the housing 102 can be formed of a material such as a metal, ceramic, polymer, composite, or the like. In some embodiments, the housing 102, or one or more portions thereof, can be formed of titanium. The header 104 can be formed of various materials, but in some embodiments the header 104 can be formed of a translucent polymer such as an epoxy material. In some embodiments the header 104 can be hollow. In other embodiments the header 104 can be filled with components and/or structural materials such as epoxy or another material such that it is non-hollow.

In some embodiments where a portion of the medical device 100 or 200 is partially external, the header 104 and housing 102 can be surrounded by a protective casing made of durable polymeric material. In other embodiments, where a portion of the medical device 100 or 200 is partially external, the header 104 and housing 102 can be surrounded by a protective casing made of a combination of polymeric material, metallic material, and/or glass material.

Header 104 can be coupled to one or more leads, such as leads 106. The header 104 can serve to provide fixation of the proximal end of one or more leads 106 and electrically couple the one or more leads 106 to one or more components within the housing 102. The one or more leads 106 can include one or more electrodes, such as electrodes 108, disposed along the length of the leads 106. In some embodiments, electrodes 108 can include electric field generating electrodes, also referred to herein as “supply electrodes.” In some embodiments electrodes 108 can include electric field sensing electrodes, also referred to herein as “sensing electrodes.”. In some embodiments, leads 106 can include both supply electrodes and sensing electrodes. In other embodiments, leads 106 can include any number of electrodes that are both supply electrodes and sensing electrodes.

It will be appreciated that while many embodiments of medical devices herein are designed to function with leads, leadless medical devices that generate electrical fields are also contemplated herein. In some embodiments, the electrodes 108 can be tip electrodes on the most distal end of the leads 106. In some embodiments, the medical devices herein can include a drug-eluting coating on the surface of the longitudinal axis of the leads in an area proximal to the cancerous tumor 110. In some embodiments, the drug-eluting coating can include an antineoplastic agent, a cytotoxic agent, or an antibiotic agent, as will be discussed in more detail below.

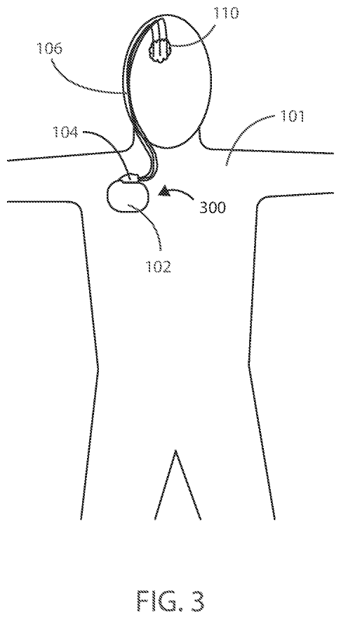

Referring now to FIG. 3, a schematic view of a patient 101 fitted with a medical device system is shown in accordance with various embodiments herein. The electrical stimulation-based cancer therapy system implanted within the patient 101 includes implantable components such as an implantable medical device 300 having a housing 102, a header 104, and leads 106. In this view, the patient 101 is shown to include a cancerous tumor 110. The leads 106 can be implanted within the patient 101 in order to interface with the cancerous tumor 110 and/or be adjacent to the cancerous tumor 110 such that electrical fields generated through the leads 106 can interface with the cancerous tumor 110.

Referring now to FIG. 4, a schematic view of a patient 101 fitted with a medical device system is shown in accordance with various embodiments herein. The electrical stimulation-based cancer therapy system implanted within the patient 101 includes implantable components such as partially implantable medical device 400 having a housing 102, a header 104, and leads 106. In this view, the patient 101 is shown to include a cancerous tumor 110. The leads 106 can be implanted within the patient 101 to interface with the cancerous tumor 110 and/or be adjacent to the cancerous tumor 110 such that electrical fields generated through the leads 106 can interface with the cancerous tumor 110.

### Leads

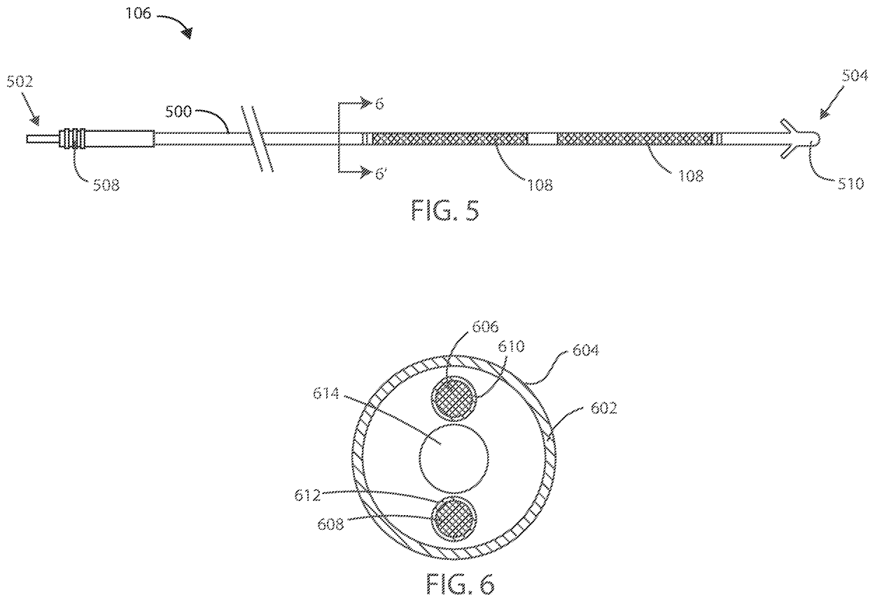

Referring now to FIG. 5, a schematic view of a lead 106 is shown in accordance with various embodiments. The lead 106 can include a lead body 500 with a proximal end 502 and a distal end 504. In various embodiments, one or more electrodes 108 can be coupled to the lead body 500. The lead body 500 can define a lumen. The lead 106 can include one or more electrodes 108 positioned near the distal end 504. The electrode 108 can include various conductive materials such as platinum, silver, gold, iridium, titanium, and various alloys. In some embodiments, the lead 106 includes more than two electrodes 108.

The lead 106 can further include a terminal pin 508 for connecting the lead 106 to an implantable device, such as a cancer treatment device. The terminal pin 508 can be compatible with various standards for lead-header interface design including the DF-1, VS-1, IS-1, LV-1 and IS-4 standards, amongst other standards.

In some embodiments, the lead 1106 can further include a fixation element 510, such as an element that can adhere to a portion of the patient's body to maintain the position of the lead 106 and/or the electrodes 108. In various embodiments, the fixation element 510 can be disposed along the distal end 504 of the lead 106. However, in some embodiments a fixation element 510 is omitted.

FIG. 6 shows a cross-sectional schematic view of a lead 106 as taken along line 6-6′ of FIG. 5. The lead 106 can include an outer layer 602 with an outer surface 604. The outer layer 602 can be flexible and can be configured to protect other components disposed within the lumen of the outer layer 602. In some embodiments, the outer layer 602 can be circular in cross-section. In some embodiments, the outer layer 602 includes a dielectric material and/or an insulator. In some embodiments, the outer layer 602 can include various biocompatible materials such as polysiloxanes, polyethylenes, polyamides, polyurethane and the like.

In various embodiments, the lead 106 can include one or more conductors, such as a first conductor 606 and a second conductor 608. In some embodiments, the first conductor 606 and the second conductor 608 can be disposed within the lumen of the outer layer 602. The first conductor 606 and a second conductor 608 can be configured to provide electrical communication between an electrode 108 and the proximal end 502 of the lead 106. The first conductor 606 and a second conductor 608 can include various materials including copper, aluminum, silver, gold, and various alloys such as tantalum/platinum, MP35N and the like. An insulator 610 and 612 can surround the first conductor 606 and a second conductor 608. The insulators 610 and 612 can include various materials such as electrically insulating polymers.

In some embodiments, each of the electrodes 108 can have individual first conductors 606 and second conductors 608 to electrically couple the electrode 108 to the proximal end 502 of the lead 106. However, in some embodiments, each of the electrodes 108 only connects to a single conductor to electrically couple the electrode 108 to the proximal end 502 of the lead 106. In some embodiments, the first conductor 606 and a second conductor 608 can be configured as a coil or a cable. Multiple conductors can be disposed within the lumen of the outer layer 602. For example, a separate conductor or set of conductors can be in communication with each electrode disposed along the lead. In various embodiments, a first conductor 606 and a second conductor 608 can form a part of an electrical circuit by which the electric fields from the electric field generating circuit are delivered to the site of the cancerous tissue. Many more conductors than are shown in FIG. 6 can be included within embodiments herein. For example, the lead 106 can include 1, 2, 3, 4, 5, 6, 7, 8, 10, 15 or 20 or more conductors, or any number of conductors falling within a range between any of the foregoing.

In some embodiments, the lead 106 can include a central channel 614. The central channel 614 can be configured for a guide wire, or other implanting device, to pass through, such as to aid in implanting the lead 106 and electrodes 108. In some cases, additional channels (not shown) are disposed within the lead 106.

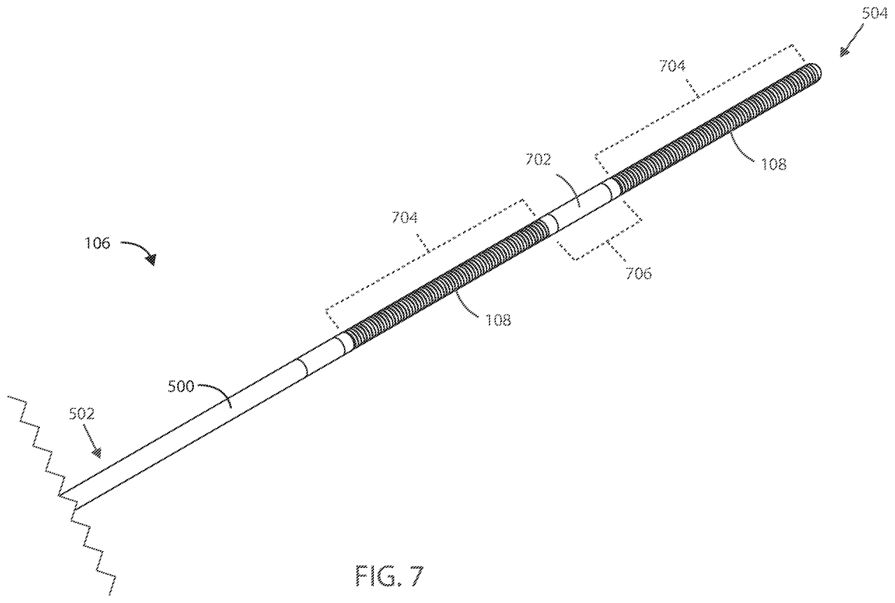

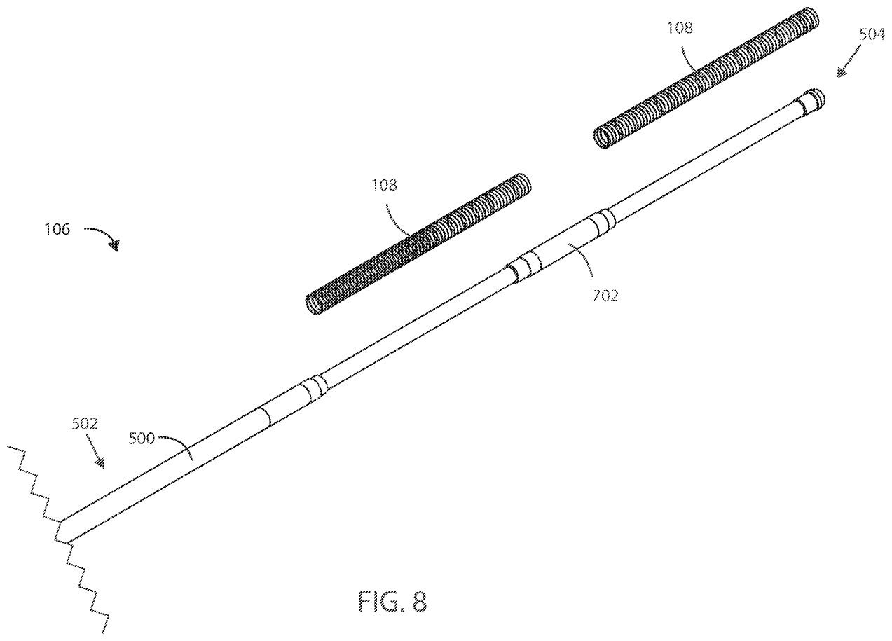

Referring now to FIG. 7 and FIG. 8, perspective views of lead 106 are shown in accordance with various embodiments. Leads 106 include one or more electrodes 108 disposed along a length of the lead body 500, where the electrodes 108 are separated by a nonconducting gap portion 702 on the exterior of the lead 106. The electrodes 108 can include an electrode length 704, and the nonconducting portion can include a non-conducting potion length of 706.

The electrode lengths 704 can each independently be from 1 cm to 4 cm in length. In some embodiments, the electrode length can be greater than or equal to 1.0 cm, 1.5 cm, 2.0 cm, 2.5 cm, 3.0 cm, 3.5 cm, 4.0 cm, 4.5 cm, 5.0 cm, 5.5 cm, or 6.0 cm, or can be an amount failing within a range between any of the foregoing. In various embodiments, when more than one electrode 108 is present along a length of the lead body, each electrode can be the same length. In various other embodiments, when more than one electrode 108 is present along a length of the lead body, each electrode can be a different length.

For leads including two or more electrodes 108, the non-conducting gap portion length 706 can be from 0.5 cm to 2 cm. In some embodiments, the non-conducing gap portion length can be greater than or equal to 0.25 cm, 0.50 cm, 0.75 cm, 1.00 cm, 1.25 cm, 1.50 cm, 1.75 cm, 2.00 cm, 2.25 cm, 2.5 cm, 2.75 cm, or 3 cm, or can be an amount falling within a range between any of the foregoing. In various embodiments, when more than one electrode 108 is present along a length of the lead body, each electrode can be separated by a non-conducting gap portion having the same length. In various other embodiments, when more than one electrode 108 is present along a length of the lead body, each electrode can be separated by a non-conducting gap portion having a different length.

### Electrodes

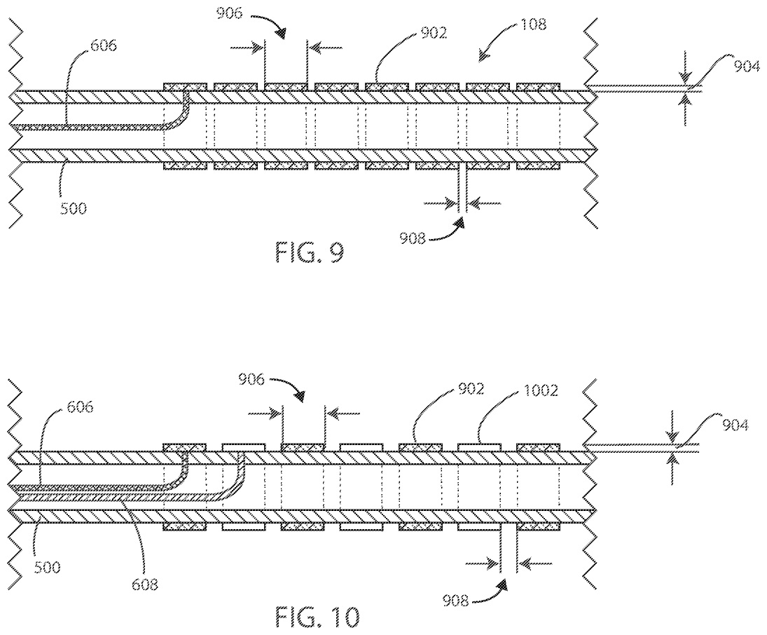

The electrodes herein are configured to generate an electric field while also being configured to reduce metal-induced magnetic resonance imaging artifacts. As discussed elsewhere herein, electrodes can include electric field generating electrodes. In various embodiments, the electrodes suitable for reducing metal-induced magnetic resonance imaging artifacts can include those such as a ribbon wire, a walled tube, or a sputter metallic electrode as will be discussed in reference to FIGS. 9-20, which show cross-sectional views of the distal most electrode 108 of the various leads embodied herein.

It should be noted that the disclosures of electrodes 108 can refer to distal most electrodes, proximal most electrodes, or any electrodes 108 disposed therebetween. In various embodiments, the electrodes herein can be disposed about the entirety of the lead body. In other embodiments, the electrodes herein can be disposed partially about the lead body, so as to only generate an electric field about a portion of the lead body. Electrodes 108 that are not the distal most can include one or more additional conductors extending through the lead body 500 and past the electrode 108, such as to electrically couple the more distal electrodes 108 to the proximal end 502 of the lead 106. In various embodiments, the electrodes herein can include those constructed of a metallic layer deposited as a sputter coating on a non-metallic substrate via a sputter coating process to achieve the thicknesses as discussed herein.

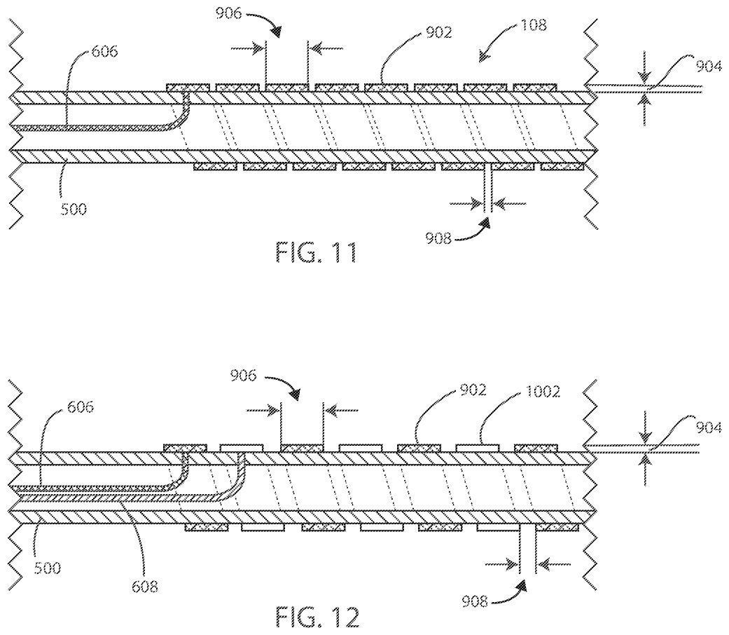

Referring now to FIGS. 9-12, cross-sectional views of various embodiments of an electrode 108 is shown are accordance with the embodiments herein. In the embodiments in FIGS. 9-12 electrode 108 can include a ribbon wire having a plurality of conductive ribbon segments disposed about the lead body 500. As shown in FIG. 9, the plurality of conductive ribbon segments can include first ribbon segments 902 that can be in electrical communication with conductor 606 and disposed circumferentially about lead body 500. As shown in FIG. 10, the plurality of conductive ribbon segments can include first ribbon segments 902 that can be in electrical communication with conductor 606 and second ribbon segments 1002 that can be in electrical communication with conductor 608 and can be disposed circumferentially about lead body 500. As shown in FIG. 11, the plurality of conductive ribbon segments can include first ribbon segments 902 that can be in electrical communication with conductor 606 and disposed helically about lead body 500. As shown in FIG. 12, the plurality of conductive ribbon segments can include first ribbon segments 902 that can be in electrical communication with conductor 606 and second ribbon segments 1002 that can be in electrical communication with conductor 608 and can be disposed helically about lead body 500.

In various embodiments, when the ribbon wires are disposed circumferentially about the lead body 500, they can include those having a circumferential conductor pattern. In various embodiments, when the ribbon wires are disposed helically about the lead body 500, they can include those having a helical conductor pattern. The circumferential conductor pattern or helical conductor pattern can have an outside diameter of from 1 millimeter (mm) to 3 mm. In some embodiments, the outside diameter can be greater than or equal to 1 mm, 1.2 mm, 1.3 mm, 1.4 mm. 1.5 mm, 1.6 mm, 1.7 mm, 1.8 min, 1.9 mm, 2.0 mm, 2.1 mm, 2.2 ram, 2.3 mm, 2.4 mm, 2.5 mm, 2.6 mm, 2.7 mm, 2.8 mm. 2.9 mm, or 3.0 mm, or can be an amount falling within a range between any of the foregoing.

The first ribbon segments 902 and second ribbon segments 1002 can have a ribbon segment thickness 904 (or a thickness in a radial direction with respect to the lead body) and a ribbon segment width 906. While not intending to be bound by theory, the thickness 904 directly impacts the size of the MRI or imaging artifact. As such, in various embodiments, the thickness 904 can be sufficiently small to avoid significantly sized MRI or imaging artifacts allowing for a better view of the tissue and/or cancerous tumor within the patient.

In various embodiments, the ribbon segment thickness 904 can be greater than or equal to 0.00001 inches to 0.005 inches. In some embodiments, the ribbon segment thickness 904 can be greater than or equal to 0.00001 inches, 0.00005 inches, 0.0001 inches, 0.0005 inches, 0.001 inches, 0.005 inches, or can be an amount falling within a range between any of the foregoing. The ribbon segment width 906 can be greater than or equal to 0.00001 inches to 0.005 inches. In some embodiments, the ribbon segment width can be greater than or equal to 0.00001 inches, 0.00005 inches, 0.0001 inches, 0.0005 inches, 0.001 inches, 0.005 inches, 0.010 inches, 0.011 inches, 0.012 inches, 0.013 inches, 0.014 inches, or 0.015 inches, or can be an amount falling within a range between any of the foregoing. It will be appreciated that the ribbon wire thickness can extend in a radial direction with respect to the lead body and the ribbon wire width can extend in a longitudinal or axial direction with respect to the lead body.

In some embodiments, the ribbon segment thickness 904 and ribbon segment width 906 of the first ribbon segments 902 and second ribbon segments 1002 can be the same. In some embodiments, the ribbon segment thickness 904 and ribbon segment width 906 of the first ribbon segments 902 and second ribbon segments 1002 can be different.

The length of the entire electrodes 108 created with ribbon wires can be greater than or equal to 0.5 centimeters (cm) to 5 cm. In some embodiments, the length of the can be greater than or equal to 0.5 cm, 1.0 cm, 1.5 cm, 2.0 cm, 2.5 cm, 3.0 cm, 3.5 cm, 4.0 cm, 4.5 cm, or 5.0 cm, or can be an amount falling within a range between any of the foregoing. In some embodiments, the length can be from 1 cm to 3 cm. In some embodiments, the electrodes 108 created with ribbon wires can have a length of about 2 cm.

An electrode ribbon wire or filar with both a small thickness and width may not have sufficient durability or structural integrity to work effectively as a supply or stimulation electrode. As such, in some embodiments, the ribbon wire or filar can have a relatively small thickness along with a larger width. As such, the total cross-sectional size of the ribbon wire or filar can still be sufficiently large so as to offer sufficient durability and/or structural integrity. In various embodiments, the ribbon wire can be sized to include an aspect ratio between a thickness and a width of the ribbon wire of from 2:4 to 2:20. In various embodiments, the ribbon wire can be sized to include an aspect ratio between a thickness and a width of the ribbon wire of from 2:4 to 2:8. In various embodiments, the ribbon wire can be sized to include an aspect ratio between a thickness and a width of the ribbon wire of from 2:10. In various embodiments where the electrodes include more than one ribbon wire, it will be appreciated that each individual ribbon wire can have an aspect ratio that is the same. In various embodiments where the electrodes include more than one ribbon wire, it will be appreciated that each individual ribbon wire can have an aspect ratio that is the different.

In some embodiments, the first ribbon segments 902 can include a pitch 908 between successive or adjacent ribbon segments, such that adjacent ribbon segments do not contact each other along the lengthwise axis of the lead body 500. In some embodiments, the pitch 908 can be greater than or equal to width of the first ribbon segments 902. In some embodiments, the pitch 908 can be greater than or equal to half the width of the first ribbon segments 902. In some embodiments, the pitch 908 can be at least twice the width of the first ribbon segments 902. In some embodiments, the pitch 908 can be at least three times the width of the first ribbon segments 902. In some embodiments, the pitch 908 can be at least four times the width of the first ribbon segments 902. In some embodiments, the pitch 908 can be at least five times the width of the first ribbon segments 902. In some embodiments, the pitch 908 can be at least ten times the width of the first ribbon segments 902. In various embodiments, the each of the first ribbon segments 902 can contact neighboring ribbon segments on either side in a direction along the longitudinal axis.

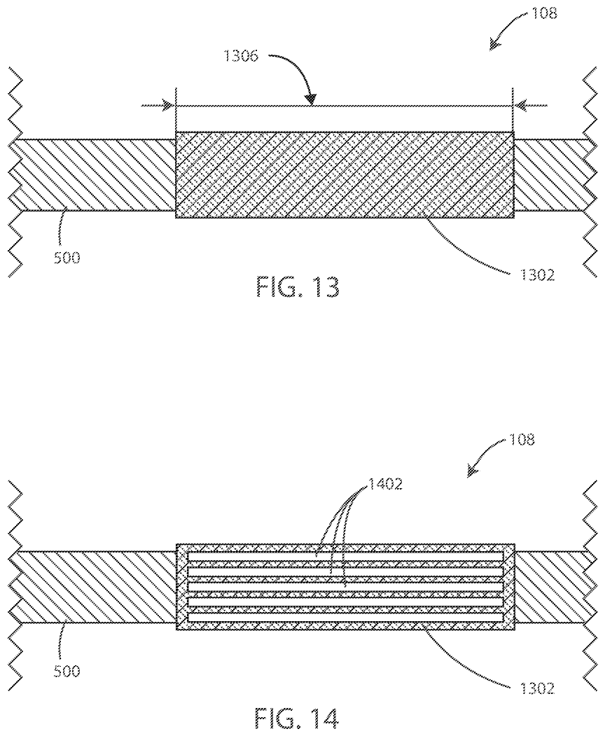

Referring now to FIGS. 13-16, schematic views of various embodiments of an electrode 108 is shown are accordance with the embodiments herein. In the embodiments in FIGS. 13-16 electrode 108 can include a walled tube segment disposed about the lead body 500. In the embodiment shown in FIG. 13, electrode 108 can include a walled tube including a conductive tubular segment 1302 disposed about a circumference of lead body 500. In some embodiments, the conductive tubular segment 1302 is disposed entirely about the circumference of the lead body 500. In other embodiments, the conductive tubular segment 1302 is disposed partially about the circumference of the lead body.

The conductive tubular segment 1302 can have a length 1306 that can be greater than or equal to 0.5 centimeters (cm) to 5 cm. In some embodiments, the length 1306 can be greater than or equal to 0.5 cm, 1.0 cm, 1.5 cm, 2.0 cm, 2.5 cm, 3.0 cm, 3.5 cm, 4.0 cm, 4.5 cm, or 5.0 cm, or can be an amount falling within a range between any of the foregoing. In some embodiments, the conductive tubular segment 1302 can have a length 1306 of from 1 cm to 3 cm. In some embodiments, the length 1306 can be about 2 cm.

The conductive tubular segments herein can include a cut-out pattern disposed therein to provide flexibility to the electrode. In the embodiment shown in FIG. 14, electrode 108 can include a conductive tubular segment 1302 that includes a cut-out pattern 1402 disposed throughout a thickness and/or length of the walled tube. In various embodiments, the conductive tubular segment 1302 includes a cut out pattern 1402 disposed partially throughout a thickness and/or length of the walled tube.

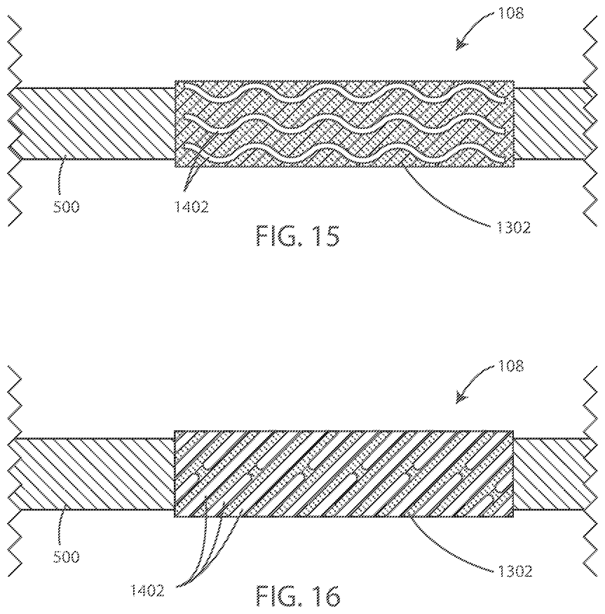

While the cut-out pattern 1402 in FIG. 14 is shown as parallel lines cut through a thickness and length about the circumference of the conductive tubular segment 1302, it will be appreciated that the cut-out pattern 1402 can assume many configurations, including but not limited to spirals, circles, squares, rectangles, triangles, hexagons, zig zags, wavy lines, and the like. By way of example, FIGS. 15 and 16 show various cut-out patterns having various configurations. In the embodiment shown in FIG. 15, electrode 108 can include a conductive tubular segment 1302 that includes a cut-out pattern 1402 configured as wavy lines disposed throughout a thickness and/or length of the walled tube. In the embodiment shown in FIG. 15, electrode 108 can include a conductive tubular segment 1302 that includes a cut-out pattern 1402 configured as discontinuous parallel lines disposed throughout a thickness and/or length of the walled tube.

In various embodiments, the cut-out pattern 1402 can extend about the entirety of the circumference of the conductive tubular segment 1302. In other embodiments, the cut-out pattern 1402 can extend partially about the circumference of the conductive tubular segment 1302. In various embodiments, the cut-out pattern can extend partially from 10%, 20%, 30%, 40%, 50%, 60%, 70%, 80%, 90%, or 100% about the circumference of the conductive tubular segment 1302. The cut-out pattern 1402 can provide flexibility to the electrode 108. In various embodiments, the conductive tubular segment 1302 does not include a cut-out pattern as shown in FIG. 13.

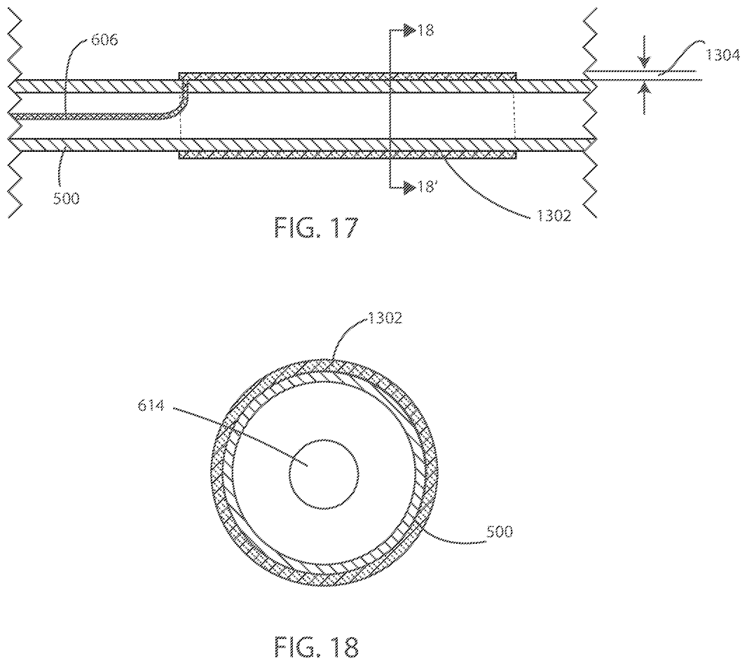

Referring now to FIG. 17, a cross-sectional view of an electrode 108 formed of a conductive tubular segment 1302 is shown in accordance with various embodiments herein. The conductive tubular segment 1302 can be in electrical communication with conductor 606 and can be disposed circumferentially about lead body 500. The conductive tubular segment 1302 can have a thickness 1304. The thickness 1304 can be greater than or equal to 0.00001 inches to 0.005 inches (or a thickness in a radial direction with respect to the lead body). In some embodiments, the thickness 1304 of the conductive tubular segment can be greater than or equal to 0.00001 inches, 0.00005 inches, 0.0001 inches, 0.0005 inches, 0.001 inches, 0.0015, 0.005 inches, 0.010 inches, 0.015 inches, 0.020 inches, 0.025 inches, 0.030 inches, or 0.035 inches 0.040 inches, 0.045 inches, or 0.05 inches, or can be an amount falling within a range between any of the foregoing.

Referring now to FIG. 18, a cross-sectional view along line 18-18′ is shown in accordance with the various embodiments herein. FIG. 18 shows the conductive tubular segment 1302 surrounding lead body 500 and a central channel 614 disposed at the center. In various embodiments the conductive tubular segment 1302 is hollow and can include a single central lumen. In various embodiments the conductive tubular segment 1302 is hollow and can include multiple lumens disposed throughout to provide passage for a guide wire, delivery of a therapeutic agent, and the like.

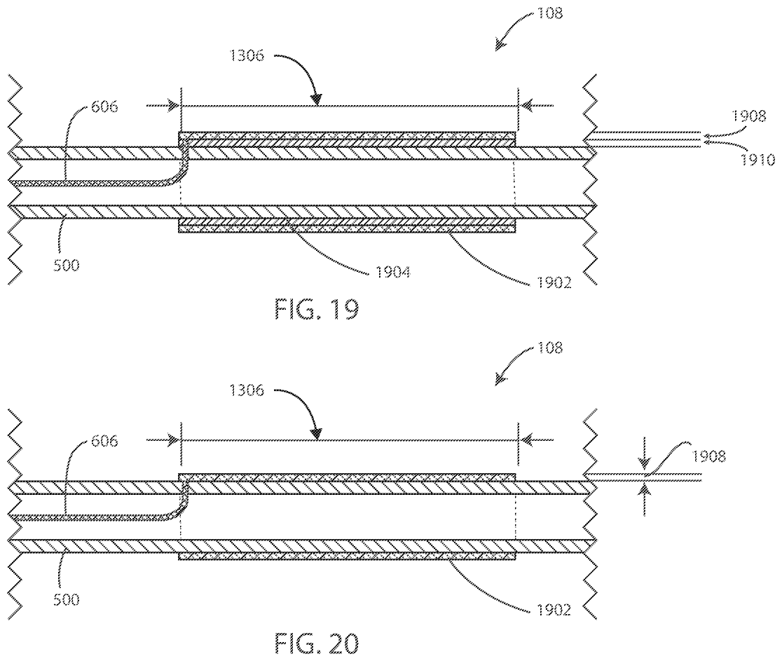

In various embodiments, the electrodes herein can include those created by the deposition of a sputter coating. Referring now to FIG. 19, a cross-sectional view of an electrode 108 including a sputter coating 1902 is shown in accordance with various embodiments herein. As shown in FIG. 19, the electrode 108 can include a sputter coating 1902 that can be in electrical communication with conductor 606 and disposed circumferentially about lead body 500. The sputter coating 1902 can be formed of a conductive metallic layer 1702. Various conductive metallic materials are discussed elsewhere herein. The sputter coating 1902 can be disposed on a non-conductive substrate 1904. The non-conductive substrate 1904 can be made from materials such as non-conductive polymers, ceramics, glasses, and the like. The length 1306 of the electrodes 108 created with a sputter coating can be greater than or equal to 0.5 centimeters (cm) to 5 cm. In some embodiments, the length 1306 can be greater than or equal to 0.5 cm, 1.0 cm, 1.5 cm, 2.0 cm, 2.5 cm, 3.0 cm, 3.5 cm, 4.0 cm, 4.5 cm, or 5.0 cm, or can be an amount falling within a range between any of the foregoing. In some embodiments, the length 1306 can be from 1 cm to 3 cm. In some embodiments, the electrode created with a sputter coating can have a length 1306 of about 2 cm.

The sputter coating 1902 can have a thickness 1908. The thickness 1908 of the sputter coating can be greater than or equal to 0.00001 inches to 0.005 inches (or a thickness in a radial direction with respect to the lead body). In some embodiments, the thickness 1908 of the sputter coating 1902 can be greater than or equal to 0.00001 inches, 0.00005 inches, 0.0001 inches, 0.0005 inches, 0.001 inches, 0.0015, 0.005 inches, 0.010 inches, 0.015 inches, 0.020 inches, 0.025 inches, 0.030 inches, or 0.035 inches 0.040 inches, 0.045 inches, or 0.05 inches, or can be an amount falling within a range between any of the foregoing.

The non-conductive substrate 1904 can have a thickness 1910. The thickness 1910 of the non-conductive substrate 1904 can be greater than or equal to 0.00001 inches to 0.005 inches (or a thickness in a radial direction with respect to the lead body). In some embodiments, the thickness 1910 of the non-conductive substrate 1904 can be greater than or equal to 0.00001 inches, 0.00005 inches, 0.0001 inches, 0.0005 inches, 0.001 inches, 0.0015, 0.005 inches, 0.010 inches, 0.015 inches, 0.020 inches, 0.025 inches, 0.030 inches, or 0.035 inches 0.040 inches, 0.045 inches, or 0.05 inches, or can be an amount falling within a range between any of the foregoing.

In various embodiments, the sputter coating can be deposited directly about the lead body 500. As shown in FIG. 20, the electrode 108 can include a sputter coating 1902 that can be in electrical communication with conductor 606 and disposed circumferentially about lead body 500. The sputter coating 1902 can be fully or partially deposited circumferentially about lead body 500 (e.g., the sputter coating 1902 can extend completely around lead body 500 or only part way). In some embodiments, the sputter coating can be deposited from 10%, 20%, 30%, 40%, 50%, 60%, 70%, 80%, 90%, or 100% circumferentially about lead body 500, or an amount falling within a range between any of the foregoing.

The ribbon wire, walled tube, and/or sputter coating electrodes herein can be formed of a conductive material (such as a conductive metal). The conductive metal can include, but is not to be limited to, one or more of copper, aluminum, silver, platinum, titanium, nickel, or a metal alloy. In some embodiments, the ribbon wire and walled tube of the electrodes can be clad with a conductive material that resists degradation such as platinum or a platinum alloy, iridium or an iridium alloy, or the like. Various techniques can be utilized to create the electrodes herein including winding the ribbon wire or walled tube into a coil, laser cutting of a walled tube, metal deposition techniques such as sputtering, and the like.

While the electrode embodiments shown in FIGS. 9-20 include one or two conductive paths, it will be appreciated that the electrodes herein can include from one to six conductive paths. In some embodiments, the electrodes herein can include greater than or equal to one, two, three, four, five, six, seven, eight, nine, or ten conducive paths, or can be an amount falling within a range between any of the foregoing. In some embodiments, the conductive paths can be arranged in parallel. In some embodiments, the conductive paths can be arranged in series.

### Medical Devices and Systems

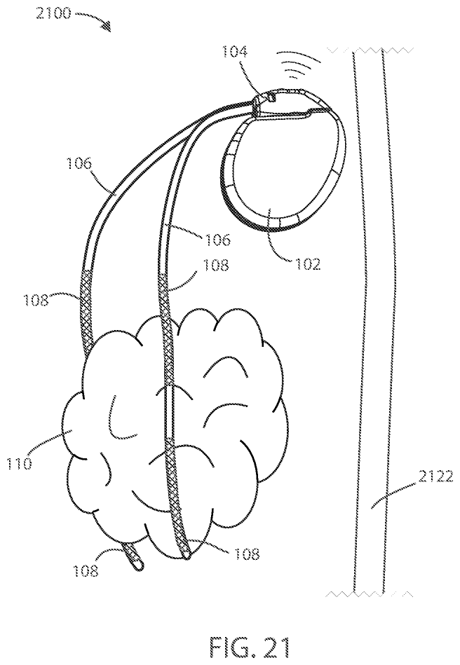

In reference now to FIG. 21, a schematic view of a medical device system for treating a cancerous tissue is shown. The medical device 2100 can include housing 102, one or more leads 106, at least one electric field generating circuit, and control circuity. The electric field generating circuit can be disposed within the housing 102. The electric field generating circuit can be configured to generate one or more electric fields. The control circuitry can be in communication with the electric field generating circuit. The control circuity can be configured to control delivery of the one or more electric fields from the electric field generating circuit. The control circuitry can cause the electric field generating circuit to generate one or more electric fields, such as at frequencies selected from a range between 10 kHz to 1 MHz, as further discussed below.

The leads 106 can include one or more implantable leads as discussed elsewhere herein. The leads 106 can include electrodes such as electrodes 108 disposed along the length of the leads 106. In various embodiments, the electrodes 108 can deliver the electric fields to the site of a cancerous tumor 110, such as a cancerous tumor, within the patient. In some embodiments, the electrodes 108 can include electric field generating electrodes and, in other embodiments, the electrodes 108 can include electric field sensing electrodes. In some embodiments, the leads 106 can include both electric field generating and electric field sensing electrodes. In various embodiments, at least one electrode 108 is configured to be implanted within the patient. In various embodiments, at least one electrode 108 is configured to be implanted entirely within or partially within the patient. In various embodiments, one or more leads 106 can be implanted leads such as first implantable lead and second implantable lead. In various embodiments, one or more electrodes 108 can be implanted electrodes. In some embodiments, at least two electrodes 108 are configured to be implanted electrodes. In other embodiments, one or more electrodes can be external electrodes.

The proximal ends (or plugs) of leads 106 can be disposed within the header 104. The distal ends of electrical leads 106 can surround a cancerous tumor 110 such that the electrodes 108 are brought into proximity of the cancerous tumor 110. In some embodiments, the leads 106 can be positioned within the vasculature such that electrodes 108 are adjacent to or positioned within the cancerous tumor 110. However, it will be appreciated that leads 106 can be disposed in various places within or around the cancerous tumor 110. In some embodiments, the leads 106 can pass directly through the cancerous tumor 110.

In some embodiments, the leads 106 can include one or more tracking markers along the length of the lead for use in determining the precise location of the electrodes relative to the tumor. In some embodiments, the one or more tracking markers can be disposed directly distal or directly proximal to the one or more electrodes disposed on the lead. In some embodiments, the tracking markers can be formed from a magnetic material. In some embodiments, the tracking markers can be formed from a radiographic material. In some embodiments, the tracking markers can be formed from a fluorographic material.

It will be appreciated that a plurality of electric field vectors can be utilized between various combinations of electrodes 108 disposed along leads 106 to create an electric field. For example, one or more electric field vectors can be generated between the most proximal electrodes 108 on the two leads 106. Similarly, one or more electric field vectors can be generated between the distal most electrodes 108 on the two leads 106. It will also be appreciated that one or more electric field vectors can be generated between any combination of electrodes 108, where various electrodes 108 can form electrode pairs. In some embodiments, one or more electric field vectors can be generated between any combination of electrodes 108 and the housing 102 of the medical device 2100. In some embodiments, the number of electric field vectors that can be generated can include greater than or equal to one, two, three, four, five, six, seven, eight, nine, or ten vectors, or can be an amount falling within a range between any of the foregoing.

It will be appreciated that one or more unipolar or multipolar leads can be used in accordance with the embodiments herein. In some embodiments, a combination of unipolar and multipolar leads can be used. In other embodiments, a circular lead, clamp lead, cuff lead, paddle lead, or patch lead can be used.

In some embodiments, a lead 106 can be a transcutaneous lead 106, such as a lead that extends through or across the skin 2122 of the patient. The tissue designated by reference number 2122 can include one or more of the epidermis, dermis, hypodermis, and/or other tissue beneath those layers. The implanted electrodes 108 can be disposed on a transcutaneous lead 106.

In some embodiments, the medical device systems herein can further include a magnetic resonance imaging (MRI) device. The MRI device can be configured to obtain one or more images of a cancerous tumor to monitor a progression or regression of the cancerous tumor.

### Therapy Parameters

The electric fields generated by the implanted medical device described herein can vary. In some embodiments, the implanted medical devices herein can generate one or more electric fields at frequencies selected from a range of between 10 kHz to 1 MHz. In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 1 MHz.

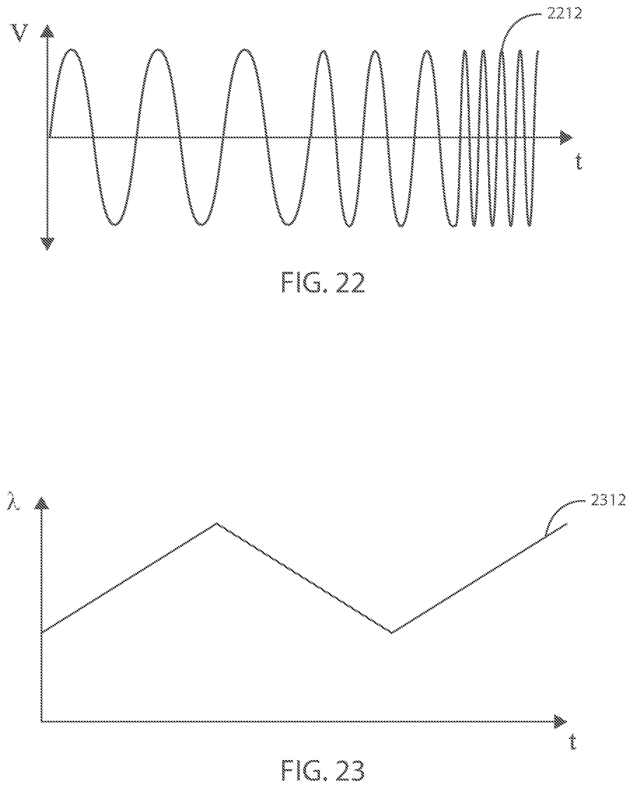

In some embodiments, an electric field can be applied to the site of a cancerous tumor at a specific frequency or constant frequency range. However, in some embodiments, an electric field can be applied to the site of a cancerous tumor by sweeping through a range of frequencies. As one example, referring now to FIG. 22, exemplary plot 2212 shows an alternating electric field, delivered by the electrodes 108, where the frequency increases over time. Similarly, FIG. 23 shows the change in frequency as a function of time in exemplary plot 2312 during a programmed therapy parameter. In some embodiments, a frequency sweep can include sweeping from a minimum frequency up to a maximum frequency. In some embodiments, a frequency sweep can include sweeping from a maximum frequency down to a minimum frequency. In other embodiments, sweeping from a minimum frequency up to a maximum frequency and sweeping from the maximum frequency down to the minimum frequency can be repeated as many times as desired throughout the duration of the delivery of the electric field from the electric field generating circuit.

As therapy progresses during a frequency sweep, it may be desired to alternate between frequency ranges so that as the cells within a population change in size and number in response to therapy, more cells can be targeted. For example, in some embodiments, a frequency sweep can include alternating between a first frequency sweep covering a range of about 100 kHz to 300 kHz and a second frequency sweep covering a range about 200 kHz to 500 kHz. It will be appreciated that sweeping through a first and second frequency range as described can be performed indefinitely throughout the course of the therapy. In some embodiments, the second frequency sweep (range) can be at higher frequencies than the first frequency sweep (range). In some embodiments, the first frequency sweep (range) can be at higher frequencies than the second frequency sweep (range).

Frequency ranges for the first and second frequency ranges can be any range including specific frequencies recited above or below, provided that the lower end of each range is a value less than the upper end of each range. At times, it may be beneficial to have some amount of overlap between the frequency range of the first and second frequency sweep.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 300 mAmp, 125 mAmp, 150 mAmp, 175 mAmp , 400 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 300 kHz, 125 kHz, 150 kHz, 175 kHz, 400 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm. In some embodiments, the system can be configured to generate one or more applied electric field strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10.0 V/cm, 20.0 V/cm, 30.0 V/cm, 40.0 V/cm, 50.0 V/cm, 60.0 V/cm, 70.0 V/cm, 80.0 V/cm, 90.0 V/cm, 300.0 V/cm, 125.0 V/cm, 150.0 V/cm, 175.0 V/cm, 400.0 V/cm, 225.0 V/cm, 250.0 V/cm, 275.0 V/cm, 300.0 V/cm, 325.0 V/cm, 350.0 V/cm, 375.0 V/cm, 400.0 V/cm, 425.0 V/cm, 450.0 V/cm, 475.0 V/cm, 500.0 V/cm, 600.0 V/cm, 700.0 V/cm, 800.0 V/cm, 900.0 V/cm, 1000.0 V/cm. It will be appreciated that the system can generate an electric field having a field strength at a treatment site falling within a range, wherein any of the foregoing field strengths can serve as the upper or lower bound of the range, provided that the upper bound is greater than the lower bound.

### Medical Device Components

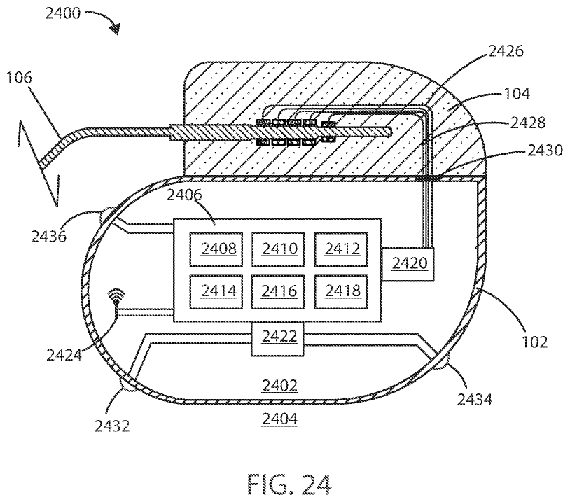

Referring now to FIG. 24, a schematic cross-sectional view of medical device 2400 is shown in accordance with various embodiments herein. The housing 102 can define an interior volume 2402 that can be hollow and that in some embodiments is hermetically sealed off from the area 2404 outside of medical device 2400. In other embodiments the housing 102 can be filled with components and/or structural materials such that it is non-hollow. The medical device 1800 can include control circuitry 2406, which can include various components 2408, 2410, 2412, 2414, 2416, and 2418 disposed within housing 102. In some embodiments, these components can be integrated and in other embodiments these components can be separate. In yet other embodiments, there can be a combination of both integrated and separate components. The medical device 2400 can also include an antenna 2424, to allow for unidirectional or bidirectional wireless data communication, such as with an external device or an external power supply. In some embodiments, the components of medical device 2400 can include an inductive energy receiver coil (not shown) communicatively coupled or attached thereto to facilitate transcutaneous recharging of the medical device via recharging circuitry.

The various components 2408, 2410, 2412, 2414, 2416, and 2418 of control circuitry 2406 can include, but are not limited to, a microprocessor, memory circuit (such as random access memory (RAM) and/or read only memory (ROM)), recorder circuitry, controller circuit, a telemetry circuit, a power supply circuit (such as a battery), a timing circuit, and an application specific integrated circuit (ASIC), a recharging circuit, amongst others. Control circuitry 2406 can be in communication with an electric field generating circuit 2420 that can be configured to generate electric current to create one or more fields. The electric field generating circuit 2420 can be integrated with the control circuitry 2406 or can be a separate component from control circuitry 2406. Control circuitry 2406 can be configured to control delivery of electric current from the electric field generating circuit 2420. In some embodiments, the electric field generating circuit 2420 can be present in a portion of the medical device that is external to the body.

In some embodiments, the control circuitry 2406 can be configured to direct the electric field generating circuit 2420 to deliver an electric field via leads 106 to the site of a cancerous tumor located within a bodily tissue. In other embodiments, the control circuitry 2406 can be configured to direct the electric field generating circuit 2420 to deliver an electric field via the housing 102 of medical device 2400 to the site of a cancerous tumor located within a bodily tissue. In other embodiments, the control circuitry 2406 can be configured to direct the electric field generating circuit 2420 to deliver an electric field between leads 106 and the housing 102 of medical device 2400. In some embodiments, one or more leads 106 can be in electrical communication with the electric field generating circuit 2420.

In some embodiments, various components within medical device 2400 can include an electric field sensing circuit 2422 configured to generate a signal corresponding to sensed electric fields. Electric field sensing circuit 2422 can be integrated with control circuitry 2406 or it can be separate from control circuitry 2406.

Sensing electrodes can be disposed on or adjacent to the housing of the medical device, on one or more leads connected to the housing, on a separate device implanted near or in the tumor, or any combination of these locations. In some embodiments, the electric field sensing circuit 2422 can include a first sensing electrode 2432 and a second sensing electrode 2434. In other embodiments, the housing 102 itself can serve as a sensing electrode for the electric field sensing circuit 2422. The electrodes 2432 and 2434 can be in communication with the electric field sensing circuit 2422. The electric field sensing circuit 2422 can measure the electrical potential difference (voltage) between the first electrode 2432 and the second electrode 2434. In some embodiments, the electric field sensing circuit 2422 can measure the electrical potential difference (voltage) between the first electrode 2432 or second electrode 2434, and an electrode disposed along the length of one or more leads 106. In some embodiments, the electric field sensing circuit can be configured to measure sensed electric fields and to record electric field strength in V/cm.

It will be appreciated that the electric field sensing circuit 2422 can additionally measure an electrical potential difference between the first electrode 2432 or the second electrode 2434 and the housing 102 itself. In other embodiments, the medical device can include a third electrode 2436, which can be an electric field sensing electrode or an electric field generating electrode. In some embodiments, one or more sensing electrodes can be disposed along lead 106 and can serve as additional locations for sensing an electric field. Many combinations can be imagined for measuring electrical potential difference between electrodes disposed along the length of one or more leads 106 and the housing 102 in accordance with the embodiments herein.

In some embodiments, the one or more leads 106 can be in electrical communication with the electric field generating circuit 2420. The one or more leads 106 can include one or more electrodes 108, as shown in FIGS. 1 and 2. In some embodiments, various electrical conductors, such as electrical conductors 2426 and 2428, can pass from the header 104 through a feed-through structure 2430 and into the interior volume 2402 of medical device 2400. As such, the electrical conductors 2426 and 2428 can serve to provide electrical communication between the one or more leads 106 and control circuitry 2406 disposed within the interior volume 2402 of the housing 102.

In some embodiments, recorder circuitry can be configured to record the data produced by the electric field sensing circuit 2422 and record time stamps regarding the same. In some embodiments, the control circuitry 2406 can be hardwired to execute various functions, while in other embodiments the control circuitry 2406 can be directed to implement instructions executing on a microprocessor or other external computation device. A telemetry circuit can also be provided for communicating with external computation devices such as a programmer, a home-based unit, and/or a mobile unit (e.g., a cellular phone, personal computer, smart phone, tablet computer, and the like).

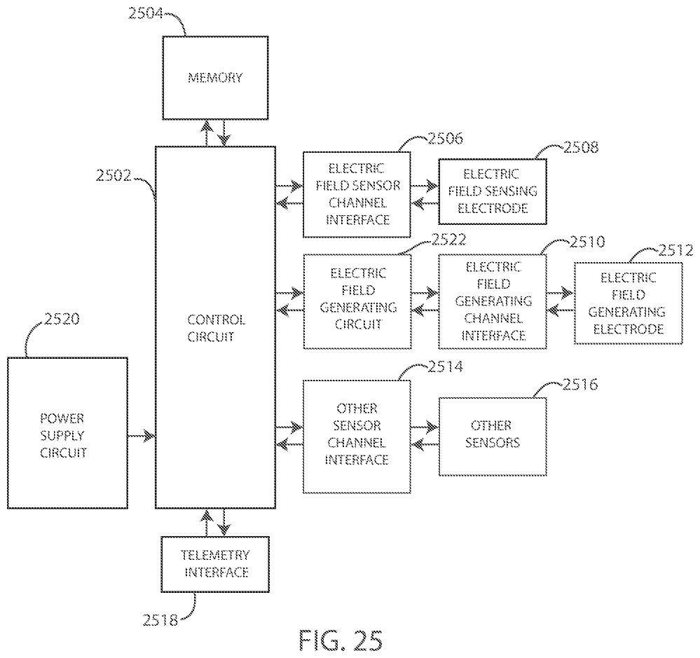

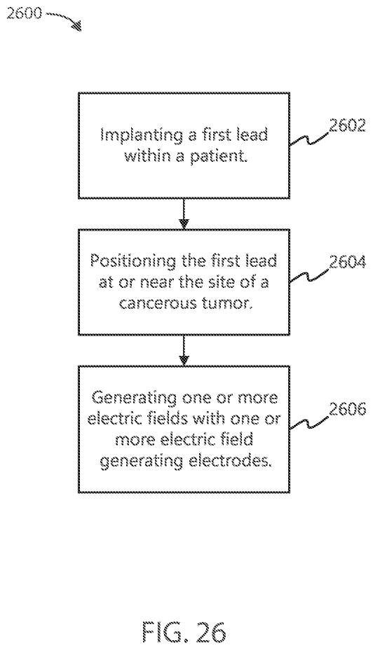

Elements of various embodiments of the medical devices described herein are shown in FIG. 25. However, it will be appreciated that some embodiments can include additional elements beyond those shown in FIG. 25. In addition, some embodiments may lack some elements shown in FIG. 25. The medical devices as embodied herein can gather information through one or more sensing channels and can output information through one or more field generating channels. A microprocessor 2502 can communicate with a memory 2504 via a bidirectional data bus. The memory 2504 can include read only memory (ROM) or random-access memory (RAM) for program storage and RAM for data storage. The microprocessor 2502 can also be connected to a telemetry interface 2518 for communicating with external devices such as a programmer, a home-based unit and/or a mobile unit (e.g., a cellular phone, personal computer, smart phone, tablet computer, and the like) or directly to the cloud or another communication network as facilitated by a cellular or other data communication network. The medical device can include a power supply circuit 2520. In some embodiments, the medical device can include an inductive energy receiver coil interface (not shown) communicatively coupled or attached thereto to facilitate transcutaneous recharging of the medical device.

The medical device can include one or more electric field sensing electrodes 2508 and one or more electric field sensor channel interfaces 2506 that can communicate with a port of microprocessor 2502. The medical device can also include one or more electric field generating circuits 2522, one or more electric field generating electrodes 2512, and one or more electric field generating channel interfaces 2510 that can communicate with a port of microprocessor 2502. The medical device can also include one or more physiological sensors, respiration sensors, or chemical sensors 2516 and one or more physiological/respiration/chemical sensor channel interfaces 2514 that can communicate with a port of microprocessor 2502. The channel interfaces 2506, 2510, and 2514 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like.

In some embodiments, the physiological sensors can include sensors that monitor temperature, blood flow, blood pressure, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

Although the physiological, respiration, or chemical sensors 2516 are shown as part of a medical device in FIG. 25, it is realized that in some embodiments one or more of the physiological, respiration, or chemical sensors could be physically separate from the medical device. In various embodiments, one or more of the physiological, respiration, or chemical sensors can be within another implanted medical device communicatively coupled to a medical device via telemetry interface 2518. In yet other embodiments, one or more of the physiological, respiration, or chemical sensors can be external to the body and coupled to a medical device via telemetry interface 2518.

### Methods

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method 2600 of treating a patient previously diagnosed with cancer is included. The method can include implanting a first lead within a patient at 2602, where the first lead can include a first lead body having a proximal end and a distal end, and one or more electric field generating electrodes disposed along a length of the lead body. The one or more electric field generating electrodes can include a ribbon wire, where a thickness of the ribbon wire in a radial direction with respect to the lead body is less than 0.005 inches. The one or more electric field generating electrodes can also include a walled tube, where a thickness of the walled tube is less than 0.005 inches. The method can further include positioning the lead at or near the site of a cancerous tumor at 2604. The method can further include generating one or more electric fields with the one or more electric field generating electrodes at 2606.

In an embodiment, the method can further include implanting a second lead within a patient.

In an embodiment of the method, the second lead comprises a second lead body can include a proximal end and a distal end, and wherein the one or more electric field generating electrodes comprise a ribbon wire, wherein a thickness of the ribbon wire in a radial direction with respect to the lead body is less than 0.005 inches, or a walled tube, wherein a thickness of the walled tube is less than 0.005 inches.

In an embodiment of the method, generating one or more electric fields with the one or more electrodes comprises generating an electrical field at a treatment site having a field strength of between 1 V/cm to 10 V/cm.

In an embodiment of the method, the one or more electric fields are delivered at one or more frequencies selected from a range of between 100 kHz to 300 kHz.

In an embodiment of the method, the one or more electric fields are generated using currents ranging from 20 mAmp to 500 mAmp.

In an embodiment of the method, the one or more electrodes forming electrode pairs defining at least two different electrical field vectors.

In an embodiment, the two different electrical field vectors angled by at least 10 degrees with respect to one another. In some embodiments, at least two vectors can be spatially separated (e.g., the vectors can be disposed at an angle with respect to one another) by at least about 10, 20, 30, 40, 50, 60, 70, 80 or 90 degrees, or can be an amount falling within a range between any of the foregoing.

In an embodiment, the method can further include obtaining magnetic resonance images of a cancerous tumor to determine a progression or regression of the cancerous tumor.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A lead for a cancer treatment system comprising:
a lead body having a proximal end and a distal end, the lead body defining a lumen; and
one or more electric field generating electrodes, wherein the one or more electric field generating electrodes are disposed along a length of the lead body;
wherein the one or more electric field generating electrodes comprise
a ribbon wire, wherein a thickness of the ribbon wire in a radial direction
with respect to the lead body is less than 0.005 inches; or
a walled tube, wherein a thickness of the walled tube is less than 0.005 inches; or
a sputter coating, wherein a thickness of the sputter coating in a radial direction with respect to a lead body is less than 0.005 inches.

2. The lead of claim 1, wherein a thickness of the ribbon wire in a radial direction with respect to the lead body is less than 0.005 inches and greater than 0.00001 inches or a thickness of the walled tube is less than 0.005 inches and greater than 0.00001 inches.

3. The lead of claim 1, wherein the one or more electric field generating electrodes comprise a ribbon wire, the ribbon wire having an aspect ratio between a thickness and a width of the ribbon of from 2:4 to 2:20.

4. The lead of claim 1, wherein the one or more electric field generating electrodes comprise a ribbon wire, the ribbon wire having an aspect ratio between a thickness and a width of the ribbon of from 2:8 to 2:10.

5. The lead of claim 1, comprising two electric field generating electrodes disposed along a length of the lead body.

6. The lead of claim 5, wherein the two electric field generating electrodes are each independently from 1 cm to 4 cm in length.

7. The lead of claim 5, wherein the two electric field generating electrodes are separated by a non-conducting gap portion of from 0.1 cm to 2 cm.

8. The lead of claim 1, wherein the one or more electric field generating electrodes comprise one or more of copper, aluminum, silver, platinum, titanium, nickel, or a metal alloy.

9. The lead of claim 1, the ribbon wire or the walled tube comprising a helical conductor pattern.

10. The lead of claim 9, wherein the helical conductor pattern has an outside diameter of 1 to 3 millimeters.

11. The lead of claim 1, wherein the one or more electric field generating electrodes comprise from one to six conductive paths arranged in parallel.

12. A method of treating a patient previously diagnosed with cancer comprising:
implanting a first lead within a patient; the first lead comprising a first lead body comprising a proximal end and a distal end; and one or more electric field generating electrodes disposed along a length of the lead body; wherein the one or more electric field generating electrodes comprise
a ribbon wire, wherein a thickness of the ribbon wire in a radial direction with respect to the lead body is less than 0.005 inches; or
a walled tube, wherein a thickness of the walled tube is less than 0.005 inches; or
a sputter coating, wherein a thickness of the sputter coating in a radial direction with respect to the lead body is less than 0.005 inches; and
positioning the lead at or near a site of a cancerous tumor; and
generating one or more electric fields with the one or more electric field generating electrodes.

13. The method of claim 12, further comprising implanting a second lead within a patient, wherein the second lead comprises a second lead body comprising a proximal end and a distal end; and
wherein the one or more electric field generating electrodes comprise
a ribbon wire, wherein a thickness of the ribbon wire in a radial direction with respect to the lead body is less than 0.005 inches; or
a walled tube, wherein a thickness of the walled tube is less than 0.005 inches.

14. The method of claim 13, wherein generating one or more electric fields with the one or more electrodes comprises generating an electrical field at a treatment site having a field strength of between 1 V/cm to 10 V/cm, at one or more frequencies selected from a range of between 100 kHz to 300 kHz, using currents ranging from 20 mAmp to 500 mAmp.

15. The method of claim 13, wherein the one or more electrodes forming electrode pairs defining at least two different electrical field vectors.

16. The method of claim 15, the two different electrical field vectors angled by at least 10 degrees with respect to one another.

17. The method of claim 12, further comprising obtaining magnetic resonance images of a cancerous tumor to determine a progression or regression of the cancerous tumor.

18. A medical device system for treating a cancerous tissue comprising:
an electric field generating circuit configured to generate one or more electric fields at or near a site of the cancerous tissue;
control circuitry in communication with the electric field generating circuit, the control circuitry configured to control delivery of the one or more electric fields from the electric field generating circuit to the site of the cancerous tissue; and
an implantable first lead, the implantable first lead comprising
a lead body having a proximal end and a distal end, the lead body defining a lumen; and
one or more electric field generating electrodes disposed along a length of the lead body, wherein the one or more electric field generating electrodes comprise
a ribbon wire, wherein a thickness of the ribbon wire in a radial direction with respect to the lead body is less than 0.005 inches; or
a walled tube, wherein a thickness of the walled tube is less than 0.005 inches; or
a sputter coating, wherein a thickness of the sputter coating in a radial direction with respect to the lead body is less than 0.005 inches.

19. The medical device system of claim 18, further comprising:
an implantable second lead, the implantable second lead comprising
a lead body having a proximal end and a distal end, the lead body defining a lumen; and
one or more electric field generating electrodes disposed along a length of the lead body,
wherein the one or more electric field generating electrodes comprise
a ribbon wire, wherein a thickness of the ribbon wire in a radial direction with respect to the lead body is less than 0.005 inches; or
a walled tube, wherein a thickness of the walled tube is less than 0.005 inches; or
a sputter coating, wherein a thickness of the sputter coating in a radial direction with respect to the lead body is less than 0.005 inches.

20. The medical device system of claim 18, wherein the control circuitry causes the electric field generating circuit to generate one or more electric fields at frequencies selected from a range of between 10 kHz to 1 MHz.
